(19) United States
(12) Patent Application Publication
(10) Pub. No.: US 2002/0115689 A1
(43) Pub. Date: Aug. 22, 2002

(54) Combination therapy for treating neurodegenerative disease

(76) Inventor: Joanne Waldstreicher, Scotch Plains, NJ (US)

Correspondence Address: MERCK AND CO INC, P O BOX 2000, RAHWAY, NJ, 070650907

(21) Appl. No.: 09/731,963

(22) Filed: Dec. 7, 2000

Related U.S. Application Data
(60) Provisional application No. 60/172,926, filed on Dec. 21, 1999.

(51) Int. Cl. A61K 31/444; A01N 43/42; A61K 31/415; A61K 31/365; A61K 31/401

(52) U.S. Cl. 514/332; 514/378; 514/406; 514/547; 514/423; 514/473

(57) ABSTRACT

The instant invention provides a novel drug combination comprised of an HMG-CoA reductase inhibitor and a selective COX-2 inhibitor, which is useful for treating, preventing, delaying the onset of and/or reducing the risk of developing Alzheimer's disease.

One object of the instant invention is to administer the above-described combination therapy to people who do not yet show clinical signs of Alzheimer's disease, but who are at risk of developing Alzheimer's disease. These individuals may already show signs of mild cognitive impairment. Toward this end, the instant invention provides methods for preventing or reducing the risk of developing Alzheimer's by administering the above-described combination therapy to said at risk persons. Such treatment may halt or reduce the rate of further cognitive decline or, in fact, reverse cognitive decline.

The present invention also provides for a method of preventing cognitive impairment or dementia, reducing the risk of cognitive decline or impairment or reducing cognitive decline or impairment resulting from stroke, stroke, cerebral ischemia or de-myelinating disorders.

### FIELD OF THE INVENTION

The instant invention involves a drug combination comprising a 3-hydroxy-3-methylglutaryl coenzyme A (HMG-CoA) reductase inhibitor and a selective inhibitor of cyclooxygenase-2 (COX-2).

### BACKGROUND OF THE INVENTION

Recently, in U.S. Pat. No. 5,840,796 the use of selective COX-2 inhibitors was disclosed for the prophylaxis and treatment of neurodegenerative diseases, including mild cognitive impairment and Alzheimer's Disease. Also mentioned are the treatment of stroke, cerebral ischemia and de-myelinating disorders, each of which may result in cognitive decline or dementia. Similarly, WO 95/006490, published Mar. 9, 1995 discloses the use of HMG-CoA reductase inhibitors such as Mevacor and Zocor in delaying the development of and preventing the onset of Alzheimer's Disease.

The list of HMG-CoA reductase inhibitors which may be used with the present invention include but are not limited to the lactonized and dihydroxy open acid forms and pharmaceutically acceptable salts and esters thereof of: lovastatin (MEVACOR®, see U.S. Pat. No. 4,342,767); simvastatin (ZOCOR®; see U.S. Pat. No. 4,444,784); pravastatin, particularly the sodium salt thereof (PRAVACHOL®; see U.S. Pat. No. 4,346,227); fluvastatin particularly the sodium salt thereof (LESCOL®; see U.S. Pat. No. 5,354,772); atorvastatin, particularly the calcium salt thereof (LIPITOR®; see U.S. Pat. No. 5,273,995); cerivastatin, particularly the sodium salt thereof (BAYCOL®, also known as rivastatin; see U.S. Pat. No. 5,177,080), nisvastatin also referred to as NK-104 (see PCT international publication number WO 97/23200) and ZD-4522 (see U.S. Pat. No. 5,260,440, and Drugs of the Future, 1999, 24(5), pp. 511-513). The structural formulas of several of these statins and additional HMG-CoA reductase inhibitors are described at page 87 of M. Yalpani, “Cholesterol Lowering Drugs”, Chemistry & Industry, pp. 85-89 (Feb. 5, 1996). HMG-CoA reductase, is an enzyme involved in the rate-limiting step in the biosynthesis of cholesterol.

The present invention provides for a method of preventing Alzheimer's disease, reducing the risk of Alzheimer's disease, delaying the onset of Alzheimer's disease and/or treating Alzheimer's disease by administering to a patient in need of such treatment a combination of an HMB-CoA reductase inhibitor such as lovastatin and simvastatin (including the open-ring dihydroxy acid forms thereof, and salts and esters thereof) and a selective inhibitor of COX-2.

Improved therapies for treating, preventing and reducing the risk of developing Alzheimer's disease are currently being sought for the large number of individuals who are at risk for this disorder. The instant invention addresses this problem by providing a combination therapy comprising an HMG-CoA reductase inhibitor with a selective inhibitor of COX-2. When administered as part of a combination therapy, the selective COX-2 inhibitor together with the HMG-CoA reductase inhibitor provide enhanced treatment options as compared to administration of either the HMG-CoA RI or the selective COX-2 inhibitor administered alone.

The present invention also provides for a method of preventing cognitive impairment or dementia, reducing the risk of cogintive decline or impairment or reducing cognitive decline or impairment resulting from stroke, multi-infarction dementia, cerebral ischemia or de-myelinating disorders.

### SUMMARY OF THE INVENTION

The instant invention provides a novel drug combination comprising an HMG-CoA reductase inhibitor and a selective COX-2 inhibitor, which combination is useful for treating, preventing, delaying the onset of and/or reducing the risk of developing Alzheimer's disease.

One object of the instant invention is to administer the above-described combination therapy to people who do not yet show clinical signs of cognitive impairment or Alzheimer's disease, but who are at risk of developing Alzheimer's disease. These individuals may already show signs of mild cognitive impairment or may be at risk of impairment due to either being homozygous or heterozygous of Apolipoprotein E isoform 4. To this end, the instant invention provides methods for preventing or reducing the risk of developing Alzheimer's by administering the above-described combination therapy to said at risk persons. Such treatment may halt or reduce the rate of further cognitive decline or, in fact, reverse cognitive decline.

A second object of the instant invention is to provide the above-described combination therapy to people who have clinical signs of cognitive impairment or Alzheimer's disease. To this end, the instant invention provides methods for halting or slowing the progression of Alzheimer's disease, by administering the above-described combination therapy to said persons who have clinically manifest Alzheimer's disease.

A third object of the instant invention involves the above-described methods further comprising the administration of one or more additional active agents either in separate or combined dosage formulations. A fourth object is to provide pharmaceutical compositions that can be used in the above-described methods. Additional objects will be evident from the following detailed description.

The present invention also provides for a method of preventing cognitive impairment or dementia, reducing the risk of cogintive decline or impairment or reducing cognitive decline or impairment resulting from stroke, stroke, cerebral ischemia or de-myelinating disorders.

### DETAILED DESCRIPTION OF THE INVENTION

The present invention provides for a method of preventing Alzheimer's disease, reducing the risk of Alzheimer's disease, delaying the onset of Alzheimer's disease and/or treating Alzheimer's disease by administering to a patient in need of such treatment a combination of an HMB-CoA reductase inhibitor such as lovastatin and simvastatin (including the open-ring dihydroxy acid forms thereof, and salts and esters thereof) and a selective inhibitor of COX-2.

In another aspect, this invention provides the use of a combination of an HMG-CoA reductase inhibitors and a selective inhibitor of COX-2 in the manufacture of a medicament for preventing Alzheimer's disease, reducing the risk of Alzheimer's disease, delaying the onset of Alzheimer's disease and/or treating Alzheimer's disease.

Thus, in one aspect the present invention provides for a method of preventing Alzheimer's disease, reducing the risk of Alzheimer's disease, delaying the onset of Alzheimer's disease and/or treating Alzheimer's disease by administering to a patient in need of such treatment a combination of an HMG-CoA reductase inhibitor such as lovastatin and simvastatin (including the open-ring dihydroxy acid forms thereof, and salts and esters thereof) and a selective inhibitor of COX-2. The present invention also provides for a method of preventing cognitive impairment or dementia, reducing the risk of cogintive decline or impairment or reducing cognitive decline or impairment resulting from stroke, stroke, cerebral ischemia or de-myelinating disorders.

The medicaments may be formulated as described in the aforementioned referenced documents. The medicament may be employed in the doses and regimens set out in the aforementioned referenced documents.

It is a great advantage of this invention that treatment may be carried out without causing gastric side effects of the type that can occur when non-selective cyclooxygenase inhibitors (NSAID's) are used for prolonged periods. Since neurodegenerative diseases such as Alzheimers disease are generally progressive, treatment may need to take place for a number of years. Thus, the provision of medicaments which are surprisingly effective without any significant tendency to cause gastric side effects at the therapeutic dose is of great use particularly to the elderly. As indicated above, the use of medicaments of this invention for the treatment of patients who are asymptotic is also envisaged especially in those cases where genetic information suggests that the patient is likely to develop Alzheimers disease.

Favourably, this invention provides a method of treating patients at risk of Alzheimer's disease without any significant tendency to cause gastric side effects which comprises the oral administration of a pharmaceutical composition which comprises an effective amount of an HMG-CoA reductase inhibitor such as lovastatin and simvastatin (including the open-ring dihydroxy acid forms thereof, and salts and esters thereof) and a selective inhibitor of COX-2 and a pharmaceutical acceptable carrier therefor.

Such a method is applicable to patients with overt symptoms of disease and is applicable to patients without overt symptoms of the disease (asymptotic patients).

Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33.

Examples of HMG-CoA reductase inhibitors that may be used include but are not limited to lovastatin (MEVACOR®; see U.S. Pat. No. 4,231,938), simvastatin (ZOCOR®; see U.S. Pat. No. 4,444,784), pravastatin (PRAVACHOL®; see U.S. Pat. No. 4,346,227), fluvastatin (LESCOL®; see U.S. Pat. No. 5,354,772), atorvastatin (LIPITOR®; see U.S. Pat. No. 5,273,995) and cerivastatin (also known as rivastatin; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (Feb. 5, 1996). The term HMG-CoA reductase inhibitor is intended to include all pharmaceutically acceptable salt, ester and lactone forms of compounds which have HMG-CoA reductase inhibitory activity, and therefor the use of such salts, esters and lactone forms is included within the scope of this invention. Preferably, the HMG-CoA reductase inhibitor is selected from lovastatin and simvastatin, and most preferably simvastatin.

In one aspect, the method of this invention is useful for treating individuals who possess one or two copies of the Apolipoprotein E type 4 allele. These individuals are more likely to develop late onset Alzheimer's disease. The method of this invention is also useful in halting the progression of Alzheimer's disease in a patient who already exhibits symptoms of dementia, and ameliorating the degenerative effects of Alzheimer's disease.

Apolipoprotein E isoform 4 (“ApoE isoform 4”) is an apolipoprotein which is the gene product of the apolipoprotein E type 4 allele. Possession of one or two copies of the apolipoprotein E type 4 allele has been linked to a greatly increased risk of developing Alzheimer's disease at longer ages (less than 65 years of age). The present invention provides for a method of decreasing circulating blood levels of ApoE isoform 4 throughout the body, including the brain. In the liver, low density lipoprotein receptors (LDL receptors) are responsible for absorbing and taking up from the bloodstream various lipoproteins including some of those containing ApoE isoform 4. LDL receptors are regulated by gene repressors derived from cholesterol, which suppress the transcription of the LDL-receptor. Inhibition of cholesterol biosynthesis reduces the presence of these cholesterol-derived LDL gene repressors. This relieves the suppression of the production of the LDL receptor, leading to production of additional LDL receptors in the liver, which, in turn, remove additional low density lipoproteins, including those containing ApoE isoform 4, from the bloodstream. Reduced levels of ApoE isoform 4 in the bloodstream promotes an increase in the flux of ApoE isoform 4 from the CNS to the plasma, thus reducing the risk of, halting the development of and/or ameliorating the symptoms of Alzheimer's disease.

Applicants also believe that HMG-CoA reductase inhibitors reduce inflammation in the brain associated with Alzheimer's disease.

In another aspect the present invention also provides for a method of preventing cognitive impairment or dementia, reducing the risk of cogintive decline or impairment or reducing cognitive decline or impairment resulting from stroke, multi-infarction dementia, cerebral ischemia or de-myelinating disorders.

As explained in J. Talley, Exp. Opin. Ther. Patents (1997), 7(1), pp. 55-62, several distinct structural classes of selective COX-2 inhibitor compounds have already been identified. One class is the methane sulfonanilide class of inhibitors, of which NS-398, flosulide, nimesulide and L-745,337 are example members.

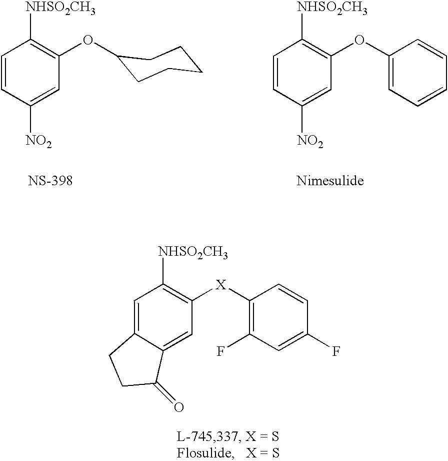

A second class is the tricyclic inhibitor class, which can be further divided into the sub-classes of tricyclic inhibitors with a central carbocyclic ring (examples include SC-57666, 1, and 2); those with a central monocyclic heterocyclic ring (examples include DuP 697, SC-58125, SC-58635, and 3, 4 and 5); and those with a central bicyclic heterocyclic ring (examples include 6, 7, 8, 9 and 10). Compounds 3, 4 and 5 are described in U.S. Pat. No. 5,474,995.

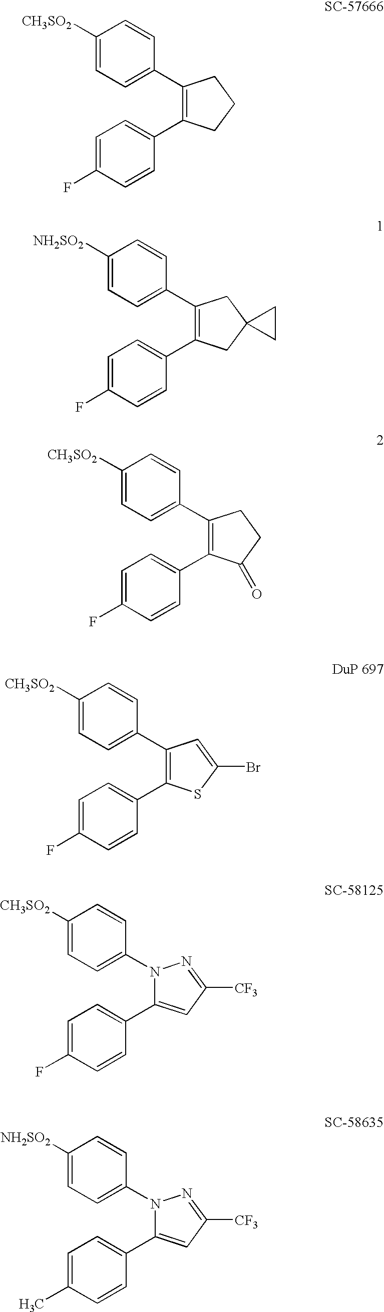

The third identified class can be referred to as those which are structurally modified NSAIDS, and includes L-761,066 and structure 11 as example members.

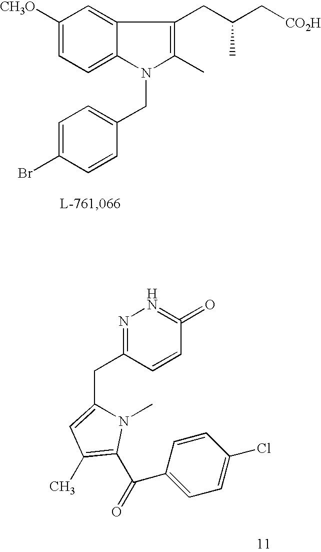

In addition to the structural classes, sub-classes, specific COX-2 inhibitor compound examples, and reference journal and patent publications described in the Talley publication which are all herein incorporated by reference, examples of compounds which selectively inhibit cyclooxygenase-2 have also been described in the following patent publications, all of which are herein incorporated by reference: U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253 , 5,604,260, 5,639,780; and International Patent Specification Nos. 94/13635, 94/15932, 94/20480, 94/26731, 94/27980, 95/00501, 95/15316, 96/03387, 96/03388, 96/06840; and International Publication No.'s WO 94/20480, WO 96/21667, WO 96/31509, WO 96/36623, WO 97/14691, WO 97/16435.

Additional COX-2 inhibitor compounds which are included in the scope of this invention include:

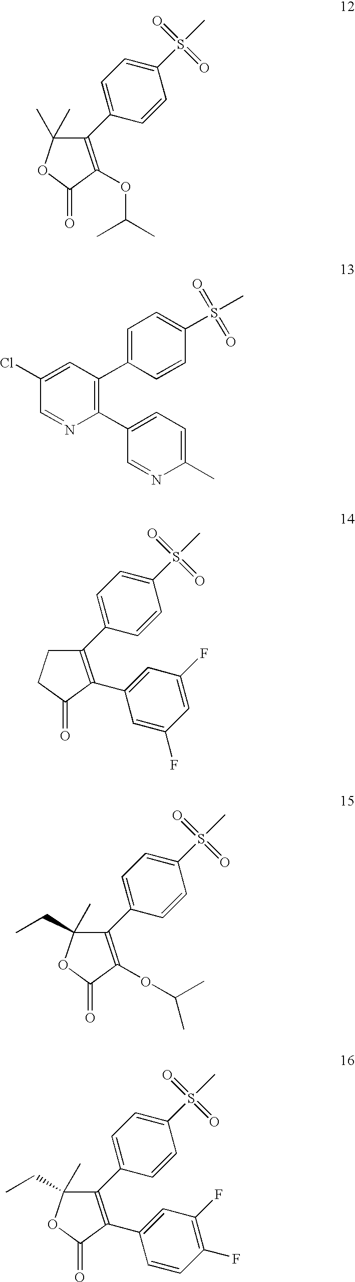

Some of the compounds above can also be identified by the following chemical names:

3: 3-phenyl-4-(4-(methylsulfonyl)phenyl)-2-(5H)-furanone;

4: 3-(3,4-difluorophenyl)-4-(4-(methylsulfonyl)phenyl)-2-(5H)-furanone;

5: 5,5-dimethyl-4-(4-(methylsulfonyl)phenyl)-3-(3-fluorophenyl)-5H-furan-2-one;

12: 5,5-dimethyl-4-(4-(methylsulfonyl)phenyl)-3-(2-propoxy)-5H-furan-2-one;

13: 5-chloro-3-(4-(methylsulfonyl)phenyl)-2-(2-methyl-5-pyridinyl)pyridine;

14: 2-(3,5-difluorophenyl)-3-(4-(methylsulfonyl)phenyl)-2-cyclopenten-1-one;

15: 5(S)-5-ethyl-5-methyl-4-(4-(methylsulfonyl)phenyl)-3-(2-propoxy)-5H-furan-2-one;

16: 5-ethyl-5-methyl-4-(4-(methylsulfonyl)phenyl)-3-(3,4-difluorophenyl)-5H-furan-2-one;

17: 3-((2-thiazolyl)methoxy)-4-(4-(methylsulfonyl)phenyl)-5,5-dimethyl-5H-furan-2-one;

18: 3-propyloxy-4-(4-(methylsulfonyl)phenyl)-5,5-dimethyl-5H-furan-2-one;

19: 3-(1-cyclopropylethoxy)-5,5-dimethyl-4-(4-methylsulfonyl)phenyl)-5H-furan-2-one;

20: sodium 2-(4-chlorophenyl)-3-(4-(methylsulfonyl)phenyl)-4-oxo-2-pentenoate;

21: 3-(cyclopropylmethoxy)-5,5-dimethyl-4-(4-(methylsulfonyl)phenyl)-5H-furan-2-one;

22: 3-(cyclopropylmethoxy)-5,5-dimethyl-4-(4-(methylsulfonyl)phenyl)-2,5-dihydrofuran-2-ol;

23: 3-isopropoxy-5,5-dimethyl-4-(4-(methylsulfonyl)phenyl)-2,5-dihydrofuran-2-ol;

24: 5,5-dimethyl-3-(3-fluorophenyl)-2-hydroxy-4-(4-(methylsulfonyl)phenyl)-2,5-dihydrofuran;

25: 5-Chloro-3-(4-(methylsulfonyl)phenyl)-2-(3-pyridinyl)pyridine.

26: 4-[5-methyl-3-phenyl-isoxazol-4-yl]benenesulfonamide;

27: N-[[4-(5-methyl-3-phenylisoxazol-4-yl)phenyl]sulfonyl]propanamide.

The following publications describe and/or provide methods for making the compounds as indicated: compounds 12, 15, 17, 18, 19 and 21, WO 97/14691; compounds 22, 23 and 24, WO 97/16435; compound 20, WO 96/36623; compound 14, U.S. Pat. No. 5,536,752; compound 16, U.S. Pat. No. 5,474,995, compound 26, U.S. Pat. No. 5,633,272; compound 27, WO 97/38986.

Also incorporated herein by reference are those compounds described in WO 96/41645 as having structural Formula, shown immediately below, and the definition and preferred definitions and species described therein:

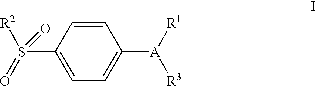

Particularly preferred compounds of formula (I) include:

5-(4-fluorophenyl)-1-[4-(methylsulfonyl)phenyl]-3-(trifluoromethyl)pyrazole;

4-(4-fluorophenyl)-5-[4-(methylsulfonyl)phenyl]-1-phenyl-3-(trifluoromethyl)pyrazole;

4-(5-(4-chlorophenyl)-3-(4-methoxyphenyl)-1H-pyrazol-1-yl)benzenesulfonamide;

4-(3,5-bis(4-methylphenyl)-1H-pyrazol-1-yl)benzenesulfonamide;

4-(5-(4-chlorophenyl)-3-phenyl-1H-pyrazol-1-yl)benzenesulfonamide;

4-(3,5-bis(4-methoxyphenyl)-1H-pyrazol-1-yl)benzenesulfonamide;

4-(5-(4-chlorophenyl)-3-(4-methylphenyl)-1H-pyrazol-1-yl)benzenesulfonamide;

4-(5-(4-chlorophenyl)-3-(4-nitrophenyl)-1H-pyrazol-1-yl)benzenesulfonamide;

4-(5-(4-chlorophenyl)-3-(5-chloro-2-thienyl)-1H-pyrazol-1-yl)benzenesulfonamide;

4-(4-chloro-3,5-diphenyl-1H-pyrazol-1-yl)benzenesulfonamide;

4-(5-(4-chlorophenyl)-3-(trifluoromethyl)-1H-pyrazol-yl)benzenesulfonamide;

4-(5-phenyl)-3-(trifluoromethyl)-1H-pyrazol-1-yl)benzenesulfonamide;

4-(5-(4-fluorophenyl)-3-(trifluoromethyl)-1H-pyrazol-1-yl)benzenesulfonamide;

4-(5-(4-methoxyphenyl)-3-(trifluoromethyl)-1H-pyrazol-1-yl)benzenesulfonamide;

4-(5-(4-chlorophenyl)-3-(difluoromethyl)-1H-pyrazol-1-yl)benzenesulfonamide;

4-(5-(4-methylphenyl)-3-(trifluoromethyl)-1H-pyrazol-1-yl)benzenesulfonamide;

4-(4-chloro-5-(4-chlorophenyl)-3-(trifluoromethyl)-1H-pyrazol-1-yl)benzenesulfonamide;

4-(3-(difluoromethyl)-5-(4-methylphenyl)-1H-pyrazol-1-yl)benzenesulfonamide;

4-(3-(difluoromethyl)-5-phenyl-1H-pyrazol-1-yl)benzenesulfonamide;

4-(3-(difluoromethyl)-5-(4-methoxyphenyl)-1H-pyrazol-1-yl)benzenesulfonamide;

4-(3-cyano-5-(4-fluorophenyl)-1H-pyrazol-1-yl)benzenesulfonamide;

4-(3-(difluoromethyl)-5-(3-fluoro-4-methoxyphenyl)-1H-pyrazol-1-yl)benzenesulfonamide;

4-(5-(3-fluoro-4-methoxyphenyl)-3-(trifluoromethyl)-1H-pyrazol-1-yl)benzenesulfonamide;

4-(4-chloro-5-phenyl-1H-pyrazol-1-yl)benzenesulfonamide;

4-(5-(4-chlorophenyl)-3-(hydroxyphenyl)-1H-pyrazol-1-yl)benzenesulfonamide;

4-(5-(4-(N,N-dimethylamino)phenyl)-3-(trifluoromethyl)-1H-pyrazol-1-yl)benzenesulfonamide;

5-(4-fluorophenyl)-6-(4-(methylsulfonyl)phenyl)spiro[2.4]hept-5-ene;

4-(6-(4-fluorophenyl)spiro[2.4]hept-5-en-5-yl)benzenesulfonamide;

6-(4-fluorophenyl)-7-(4-(methylsulfonyl)phenyl)spiro[3.4]oct-6-ene;

5-(3-chloro-4-methoxyphenyl)-6-(4-(methylsulfonyl)phenyl)spiro[2.4]hept-5-ene;

4-(6-(3-chloro-4-methoxyphenyl)spiro[2.4]hept-5-en-5-yl)benzenesulfonamide;

5-(3,5-dichloro-4-methoxyphenyl)-6-(4-(methylsulfonyl)phenyl)spiro[2.4]hept-5-ene;

5-(3-chloro-4-fluorophenyl)-6-(4-(methylsulfonyl)phenyl)spiro[2.4]hept-5-ene;

4-(6-(3,4-dichlorophenyl)spiro[2.4]hept-5-en-5-yl)benzenesulfonamide;

2-(3-chloro-4-fluorophenyl)-4-(4-fluorophenyl)-5-(4-methylsulfonylphenyl)thiazole;

2-(2-chlorophenyl)-4-(4-fluorophenyl)-5-(4-methylsulfonylphenyl)thiazole;

5-(4-fluorophenyl)-4-(4-methylsulfonylphenyl)-2-methylthiazole;

4-(4-fluorophenyl)-5-(4-methylsulfonylphenyl)-2-trifluoromethylthiazole;

4-(4-fluorophenyl)-5-(4-methylsulfonylphenyl)-2-(2-thienyl)thiazole;

4-(4-fluorophenyl)-5-(4-methylsulfonylphenyl)-2-benzylaminothiazole;

4-(4-fluorophenyl)-5-(4-methylsulfonylphenyl)-2-(1-propylamino)thiazole;

2-((3,5-dichlorophenoxy)methyl)-4-(4-fluorophenyl)-5-(4-(methylsulfonyl)phenyl)thiazole;

5-(4-fluorophenyl)-4-(4-methylsulfonylphenyl)-2-trifluoromethylthiazole;

1-methylsulfonyl-4-(1,1-dimethyl-4-(4-fluorophenyl)cyclopenta-2,4-dien-3-yl)benzene;

4-(4-(4-fluorophenyl)-1,1-dimethylcyclopenta-2,4-dien-3-yl)benzenesulfonamide;

5-(4-fluorophenyl)-6-(4-(methylsulfonyl)phenyl)spiro[2.4]hepta-4,6-diene;

4-(6-(4-fluorophenyl)spiro[2.4]hepta-4,6-dien-5-yl)benzenesulfonamide;

6-(4-fluorophenyl)-2-methoxy-5-(4-(methylsulfonyl)phenyl)-pyridine-3-carbonitrile;

2-bromo-6-(4-fluorophenyl)-5-(4-(methylsulfonyl)phenyl)-pyridine-3-carbonitrile;

6-(4-fluorophenyl)-5-(4-(methylsulfonyl)phenyl)-2-phenyl-pyridine-3-carbonitrile;

4-(2-(4-methylpyridin-2-yl)-4-(trifluoromethyl)-1H-imidazol-1-yl)benzenesulfonamide;

4-(2-(5-methylpyridin-3-yl)-4-(trifluoromethyl)-1H-imidazol-1-yl)benzenesulfonamide;

4-(2-(2-methylpyridin-3-yl)-4-(trifluoromethyl)-1H-imidazol-1-yl)benzenesulfonamide;

3-(1-(4-(methylsulfonyl)phenyl)-4-(trifluoromethyl)-1H-imidazol-2-yl)benzenesulfonamide;

2-(1-(4-(methylsulfonyl)phenyl)-4-(trifluoromethyl)-1H-imidazol-2-yl)pyridine;

2-methyl-4-(1-(4-(methylsulfonyl)phenyl)-4-(trifluoromethyl)-1H-imidazol-2-yl)pyridine;

2-methyl-6-(1-(4-(methylsulfonyl)phenyl)-4-(trifluoromethyl)-1H-imidazol-2-yl)pyridine;

4-(2-(6-methylpyridin-3-yl)-4-(trifluoromethyl)-1H-imidazol-1-yl)benzenesulfonamide;

2-(3,4-difluorophenyl)-1-(4-(methylsulfonyl)phenyl)-4-(trifluoromethyl)-1H-imidazole;

4-(2-(4-methylphenyl)-4-(trifluoromethyl)-1H-imidazol-1-yl)benzenesulfonamide;

2-(4-chlorophenyl)-1-(4-(methylsulfonyl)phenyl)-4-methyl-1H-imidazole;

2-(4-chlorophenyl)-1-(4-(methylsulfonyl)phenyl)-4-phenyl-1H-imidazole;

2-(4-chlorophenyl)-4-(4-fluorophenyl)-1-(4-(methylsulfonyl)phenyl)-1H-imidazole;

2-(3-fluoro-4-methoxyphenyl)-1-(4-(methylsulfonyl)phenyl)-4-(trifluoromethyl)-1H-imidazole;

1-(4-(methylsulfonyl)phenyl)-2-phenyl-4-trifluoromethyl-1H-imidazole;

2-(4-methylphenyl)-1-(4-(methylsulfonyl)phenyl)-4-trifluoromethyl-1H-imidazole;

4-(2-(3-chloro-4-methylphenyl)-4-(trifluoromethyl)-1H-imidazol-1-yl)benzenesulfonamide;

2-(3-fluoro-5-methylphenyl)-1-(4-(methylsulfonyl)phenyl)-4-(trifluoromethyl)-1H-imidazole;

4-(2-(3-fluoro-5-methylphenyl)-4-(trifluoromethyl)-1H-imidazol-1-yl)benzenesulfonamide;

2-(3-methylphenyl)-1-(4-(methylsulfonyl)phenyl)-4-(trifluoromethyl)-1H-imidazole;

4-(2-(3-methylphenyl)-4-(trifluoromethyl)-1H-imidazol-1-yl)benzenesulfonamide;

1-(4-(methylsulfonyl)phenyl)-2-(3-chlorophenyl)-4-(trifluoromethyl)-1H-imidazole;

4-(2-(3-chlorophenyl)-4-(trifluoromethyl)-1H-imidazol-1-yl)benzenesulfonamide;

4-(2-phenyl-4-(trifluoromethyl)-1H-imidazol-1-yl)benzenesulfonamide;

4-(2-(4-methoxy-3-chlorophenyl)-4-(trifluoromethyl)-1H-imidazol-1-yl)benzenesulfonamide;

1-allyl-4-(4-fluorophenyl)-3-(4-(methylsulfonyl)phenyl)-5-(trifluoromethyl)-1H-pyrazole;

4-(1-ethyl-4-(4-fluorophenyl)-5-(trifluoromethyl)-1H-pyrazol-3-yl)benzenesulfonamide;

N-phenyl-(4-(4-fluorophenyl)-3-(4-(methylsulfonyl)phenyl)-5-(trifluoromethyl)-1H-pyrazol-1-yl)acetamide;

ethyl(4-(4-fluorophenyl)-3-(4-(methylsulfonyl)phenyl)-5-(trifluoromethyl)-1H-pyrazol-1-yl)acetate;

4-(4-fluorophenyl)-3-(4-(methylsulfonyl)phenyl)-1-(2-phenylethyl)-1H-pyrazole;

4-(4-fluorophenyl)-3-(4-(methylsulfonyl)phenyl)-1-(2-phenylethyl)-5-(trifluoromethyl)pyrazole;

1-ethyl-4-(4-fluorophenyl)-3-(4-(methylsulfonyl)phenyl)-5-(trifluoromethyl)-1H-pyrazole;

5-(4-fluorophenyl)-4-(4-(methylsulfonyl)phenyl)-2-(trifluoromethyl)-1H-imidazole;

4-(4-(methylsulfonyl)phenyl)-5-(2-thiophenyl)-2-(trifluoromethyl)-1H-imidazole;

5-(4-fluorophenyl)-2-methoxy-4-(4-(methylsulfonyl)phenyl)-6-(trifluoromethyl)pyridine;

2-ethoxy-5-(4-fluorophenyl)-4-(4-(methylsulfonyl)phenyl)-6-(trifluoromethyl)pyridine;

5-(4-fluorophenyl)-4-(4-(methylsulfonyl)phenyl)-2-(2-propynyloxy)-6-(trifluoromethyl)pyridine;

2-bromo-5-(4-fluorophenyl)-4-(4-(methylsulfonyl)phenyl)-6-(trifluoromethyl)pyridine;

4-(2-(3-chloro-4-methoxyphenyl)-4,5-difluorophenyl)benzenesulfonamide;

1-(4-fluorophenyl)-2-(4-(methylsulfonyl)phenyl)benzene;

5-difluoromethyl-4-(4-(methylsulfonyl)phenyl)-3-phenylisoxazole;

4-(3-ethyl-5-phenylisoxazol-4-yl)benzenesulfonamide;

4-(5-difluoromethyl-3-phenylisoxazol-4-yl)benzenesulfonamide;

4-(5-hydroxymethyl-3-phenylisoxazol-4-yl)benzenesulfonamide;

4-(5-methyl-3-phenylisoxazol-4-yl)benzenesulfonamide;

1-(2-(4-fluorophenyl)cyclopenten-1-yl)-4-(methylsulfonyl)benzene;

1-(2-(4-fluoro-2-methylphenyl)cyclopenten-1-yl)-4-(methylsulfonyl)benzene;

1-(2-(4-chlorophenyl)cyclopenten-1-yl)-4-(methylsulfonyl)benzene;

1-(2-(2,4-dichlorophenyl)cyclopenten-1-yl)-4-(methylsulfonyl)benzene;

1-(2-(4-trifluoromethylphenyl)cyclopenten-1-yl)-4-(methylsulfonyl)benzene;

1-(2-(4-methylthiophenyl)cyclopenten-1-yl)-4-(methylsulfonyl)benzene;

1-(2-(4-fluorophenyl)-4,4-dimethylcyclopenten-1-yl)-4-(methylsulfonyl)benzene;

4-(2-(4-fluorophenyl)-4,4-dimethylcyclopenten-1-yl)benzenesulfonamide;

1-(2-(4-chlorophenyl)-4,4-dimethylcyclopenten-1-yl)-4-(methylsulfonyl)benzene;

4-(2-(4-chlorophenyl)-4,4-dimethylcyclopenten-1-yl)benzenesulfonamide;

4-(2-(4-fluorophenyl)cyclopenten-1-yl)benzenesulfonamide;

4-(2-(4-chlorophenyl)cyclopenten-1-yl)benzenesulfonamide;

1-(2-(4-methoxyphenyl)cyclopenten-1-yl)-4-(methylsulfonyl)benzene;

1-(2-(2,3-difluorophenyl)cyclopenten-1-yl)-4-(methylsulfonyl)benzene;

4-(2-(3-fluoro-4-methoxyphenyl)cyclopenten-1-yl)benzenesulfonamide;

1-(2-(3-chloro-4-methoxyphenyl)cyclopenten-1-yl)-4-(methylsulfonyl)benzene;

4-(2-(3-chloro-4-fluorophenyl)cyclopenten-1-yl)benzenesulfonamide;

4-(2-(2-methylpyridin-5-yl)cyclopenten-1-yl)benzenesulfonamide; ethyl 2-(4-(4-fluorophenyl)-5-(4-(methylsulfonyl)phenyl)oxazol-2-yl)-2-benzyl-acetate;

2-(4-(4-fluorophenyl)-5-(4-(methylsulfonyl)phenyl)oxazol-2-yl)acetic acid;

2-(tert-butyl)-4-(4-fluorophenyl)-5-(4-(methylsulfonyl)phenyl)oxazole;

4-(4-fluorophenyl)-5-(4-(methylsulfonyl)phenyl)-2-phenyloxazole;

4-(4-fluorophenyl)-2-methyl-5-(4-(methylsulfonyl)phenyl)oxazole; and

4-(5-(3-fluoro-4-methoxyphenyl)-2-trifluoromethyl-4-oxazolyl)benzenesulfonamide;

or a pharmaceutically acceptable salt thereof.

The ability of a compound to inhibit COX-1 and/or COX-2 may be characterized by reference to a wide variety of assays. However, for purposes of this patent application COX-1 and COX-2 inhibition and the selective inhibition of cyclooxygenase-2, such as in the term “selective inhibitor of COX-2” shall be defined by reference to the human whole blood assay as desclosed in Chan, et. al., JPET 290:551-560, 1999 assay, herein incorporated by reference. Thus, a selective inhibitor of COX-2 as used herein embrace compounds which possess an IC₅₀ for the inhibition of COX-2 that is smaller than it's IC₅₀ for the inhibit COX-1 as measured by the Chan, et. al., human whole blood assay. Moreover, for purposes of this patent application a COX-2 selectivity ratio is defined by the IC₅₀ for the inhibition of COX-1 divided by the IC₅₀ for the inhibition of COX-2 as measured by the Chan, et al., human whole blood assay. The following Table is illustrative of the approximate COX-2 selectivity ratio's for known compounds:

| NSAID | COX-1/COX-2 ratio of the IC₅₀ |
|---|---|
| Rofecoxib (Compound 3) | 35. |
| Compound 13 | 80 |
| Celecoxib (SC-58635) | 6.5 |
| Valdecoxib (Compound 26) | 30. |
| Pericoxib (Compound 27) | >100 |
| Meloxicam | 2. |
| Diclofenac | 3. |
| Indomethacin | 0.4 |

Preferably, the compounds have a selectivity ratio of cyclooxygenase-2 inhibition over cyclooxygenase-1 inhibition of at least 5, more preferably of at least 20. The resulting selectivity may indicate an ability to reduce the incidence of common NSAID-induced side effects.

The selective COX-2 inhibitors for use in this invention are most aptly those which are highly brain penetrant so that the maximum concentration of selective inhibitor of COX-2 after administration of the effective dose of selective COX-2 inhibitor is at least the IC₅₀ value.

The instant pharmaceutical combination comprising an HMG-CoA reductase inhibitor in combination with a COX-2 inhibitor includes administration of a single pharmaceutical dosage formulation which contains both the HMG-CoA reductase inhibitor and the COX-2 inhibitor, as well as administration of each active agent in its own separate pharmaceutical dosage formulation. Where separate dosage formulations are used, the HMG-CoA reductase inhibitor and the COX-2 inhibitor can be administered at essentially the same time, i.e., concurrently, or at separately staggered times, i.e, sequentially. The instant pharmaceutical combination is understood to include all these regimens. Administration in these various ways are suitable for the present invention as long as the beneficial pharmaceutical effect of the HMG-CoA reductase inhibitor and the COX-2 inhibitor are realized by the patient at substantially the same time. It is preferred that the HMG-CoA reductase inhibitor and the COX-2 inhibitor be co-administered concurrently on a once-a-day dosing schedule; however, varying dosing schedules, such as the HMG-CoA reductase inhibitor once per day and the COX-2 inhibitor once, twice or more times per day, is also encompassed herein. A single oral dosage formulation comprised of both an HMG-CoA reductase inhibitor and the COX-2 inhibitor is preferred. A single dosage formulation will provide convenience for the patient, which is an important consideration especially for patients who already have coronary heart disease and may be in need of multiple medications.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. The dosage regimen utilizing an HMG-CoA reductase inhibitor in combination with COX-2 inhibitor is selected in accordance with a variety of factors including type, species, age, weight, sex and medical condition of the patient: the severity of the condition to be treated; the route of administration; the renal and hepatic function of the patient; and the particular compound or salt or ester thereof employed. Since two different active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts needed to prevent, counter, or arrest the progress of the condition.

The term “patient” includes mammals, especially humans, who take an HMG-CoA reductase inhibitor in combination with a COX-2 inhibitor for any of the uses described herein. Administering of the drug combination to the patient includes both self-administration and administration to the patient by another person.

The daily dosage amounts of the HMG-CoA reductase inhibitor are intended to be the same or similar to those amounts which are employed for anti-hypercholesterolemic treatment and which are described in the Physicians' Desk Reference (PDR). For example, see the 50^{th} Ed. of the PDR, 1996 (Medical Economics Co); in particular, see at page 216 the heading “Hypolipidemics,” sub-heading “HMG-CoA Reductase Inhibitors,” and the reference pages cited therein. Preferably, the oral dosage amount of HMG-CoA reductase inhibitor is from about 1 to 200 mg/day, and more preferably from about 5 to 160 mg/day. However, dosage amounts will vary depending on the potency of the specific HMG-CoA reductase inhibitor used as well as other factors as noted above. An HMG-CoA reductase inhibitor which has sufficiently greater potency may be given in sub-milligram daily dosages. The HMG-CoA reductase inhibitor may be administered from 1 to 4 times per day, and preferably once per day.

As examples, the daily dosage amount for simvastatin may be selected from 5 mg, 10 mg, 20 mg, 40 mg, 80 mg and 160 mg; for lovastatin, 10 mg, 20 mg, 40 mg and 80 mg; for fluvastatin sodium, 20 mg, 40 mg and 80 mg; for pravastatin sodium, 10 mg, 20 mg, and 40 mg; and for atorvastatin calcium, 10 mg, 20 mg, and 40 mg.

The inhibitor of cyclooxygenase-2 may be administered at a dosage level up to conventional dosage levels for NSAIDs. Suitable dosage levels will depend upon the antiinflammatory effect of the chosen inhibitor of cyclooxygenase-2, but typically suitable levels will be about 0.001 to 50 mg/kg per day, preferably 0.005 to 30 mg/kg per day, and especially 0.05 to 10 mg/kg per day. The compound may be administered on a regimen of up to 6 times per day, preferably 1 to 4 times per day, and especially once per day.

Additional active agents may be used in combination with the HMG-CoA reductase inhibitor and COX-2 inhibitor in a single dosage formulation, or may be administered to the patient in a separate dosage formulation, which allows for concurrent or sequential administration. One or more additional active agents may be administered with the HMG-CoA reductase inhibitor and COX-2 inhibitor. The additional active agent or agents can be cholesterol lowering compounds. Examples of additional active agents which may be employed include HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors; probucol; niacin; fibrates such as clofibrate, fenofibrate, and gemfibrizol; cholesterol absorption inhibitors; bile acid sequestrants; LDL (low density lipoprotein) receptor inducers; platelet aggregation inhibitors, for example glycoprotein IIb/IIIa fibrinogen receptor antagonists and aspirin; vitamin B₆ (also known as pyridoxine) and the pharmaceutically acceptable salts thereof such as the HCl salt; vitamin B₁₂ (also known as cyanocobalamin); beta-blockers; folic acid or a pharmaceutically acceptable salt or ester thereof such as the sodium salt and the methylglucamine salt; and anti-oxidant vitamins such as vitamin C and E and beta carotene. The additional active agents may also include cholinesterase inhibitors such as donepezil (ARICEPT), EXELON (rivistigmine) and COGNEX tacrine HCL)).

Examples of HMG-CoA synthase inhibitors include: the beta-lactone derivatives disclosed in U.S. Pat. Nos. 4,806,564, 4,816,477, 4,847,271, and 4,751,237; the beta lactam derivatives disclosed in U.S. Pat. No. 4,983,597 and the substituted oxacyclopropane analogues disclosed in European Patent Publication EP O 411 703. The squalene synthetase inhibitors suitable for use herein include, but are not limited to, those disclosed by Biller et al., J. Med. Chem., 1988 Vol. 31, No. 10, pp. 1869-1871, including isoprenoid (phosphinylmethyl)-phosphonates such as those of the formula.

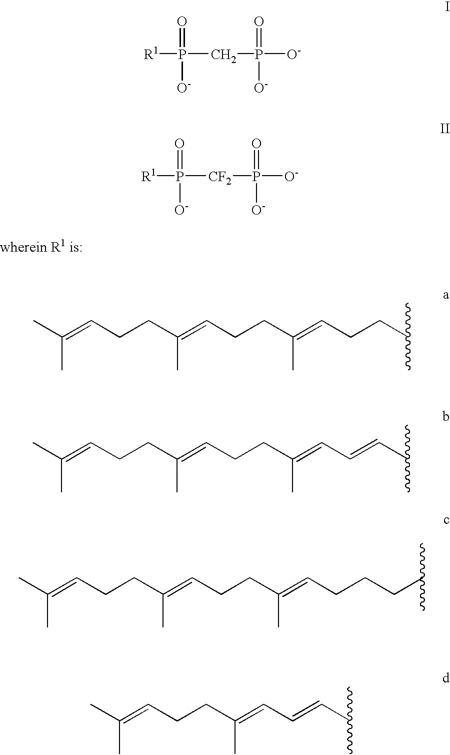

including the triacids thereof, triesters thereof and tripotassium and trisodium salts thereof as well as other squalene synthetase inhibitors disclosed in pending U.S. Pat. Nos. 4,871,721 and 4,924,024 and in Biller et al., J. Med.Chem., 1988, Vol. 31, No. 10, pp. 1869 to 1871.

In addition, other squalene synthetase inhibitors suitable for use herein include the terpenoid pyrophosphates disclosed by P. Ortiz de Montellano et al., J. Med. Chem., 1977, 20, 243-249, the farnesyl diphosphate analog A and presqualene pyrophosphate (PSQ-PP) analogs as disclosed by Corey and Volante, J. Am. Chem. Soc. 1976, 98, 1291-1293, phosphinylphosphonate reported by McClard, R. W. et al., J.A.C.S., 1987, 109, 5544 and cyclopropanes reported by Capson, T. L., PhD dissertation, June, 1987, Dept. Med. Chem. U. of Utah, Abstract, Table of Contents, pp. 16, 17, 40-43, 48-51, Summary.

Further, the benzodiazepine squalene synthase inhibitors described in EP O 567 026 to Takeda Chemical Industries, and the quinuclidinyl squalene synthase inhibitors described in PCT publications WO 94/03451, WO 93/09115, WO 93/21183, WO 93/21184, WO 93/24486, and U.S. Pat. No. 5,135,935, may be co-administered with the HMG-CoA RI plus COX-2 inhibitor combination of the present invention. In addition, the zaragozic acid type squalene synthase inhibitors as described in U.S. Pat. Nos. 5,284,758; 5,283,256; 5,262,435; 5,260,332; 5,264,593; 5,260,215; 5,258,401; 5,254,727; 5,256,689; 5,132,320; 5,278,067, and PCT Publications WO 92/12156; WO 92/12157; WO 92/12158; WO 92/12159; WO 92/12160; WO 93/18040; WO 93/18039; WO 93/07151; and European Patent Publications EP O 512 865, EP O 568 946; EP O 524,677 and EP O 450 812, as well as the acyclic tricarboxylic acid compounds of U.S. Pat. No. 5,254,727, may be employed.

Illustrative examples of squalene epoxidase inhibitors are disclosed in European Patent Publication EP O 318 860 and in Japanese Patent Publication JO2 169-571A. LDL-receptor gene inducer molecules are disclosed in U.S. Pat. No. 5,182,298.

Examples of bile acid sequestrants which may be employed in the present method include cholestyramine, colestipol, and poly[methyl-(3-trimethylaminopropyl)imino-trimethylene dihalide] and those disclosed in WO95/34585 to Geltex Pharmaceuticals, Inc. and EP 0 622 078 assigned to Hisamitsu Pharmaceutical Co., Inc.

Examples of cholesterol absorption inhibitors which may be employed in the present method include those described in WO 95/18143 and WO 95/18144 both assigned to Pfizer Inc., and WO 94/17038, WO 95/08532 and WO 93/02048 each assigned to Schering Corp.

The additional active agents described above which may be employed along with the HMG-CoA reductase inhibitor and COX-2 inhibitor combination therapy can be used, for example, in amounts as indicated in the PDR or in amounts as indicated in the reference disclosures, as appropriate.

The active agents employed in the instant combination therapy can be administered in such oral forms as tablets, capsules (each of which includes sustained release or timed release formulations), pills, powders, granules, elixirs, tinctures, suspensions, syrups, and emulsions. The instant invention includes the use of both oral rapid-release and time-controlled release pharmaceutical formulations. A particular example of an oral time-controlled release pharmaceutical formulation is described in U.S. Pat. No. 5,366,738. Oral formulations are preferred. Such pharmaceutical compositions are known to those of ordinary skill in the pharmaceutical arts; for example, see Remington's Pharmaceutical Sciences, Mack Publishing Co., Easton, Pa.

In the methods of the present invention, the active agents are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

The active agents of the present invention may be administered orally, topically, parenterally, by inhalation spray or rectally in dosage unit formulations containing conventional non-toxic pharmaceutically acceptable carriers. It is usually desirable to use the oral route. The active agents reductase inhibitor may be administered orally in the form of a capsule, a tablet or the like. The orally administered medicament may be administered in the form of a time-controlled release vehicle, including diffusion-controlled systems, osmotic devices, dissolution-controlled matrices and erodible/degradable matrices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants (BHA, BHT, propyl gallate, sodium ascorbate, citric acid) can also be added to stabilize the dosage forms. Other suitable components include gelatin, sweeteners, natural and synthetic gums such as acacia, tragacanth or alginates, carboxymethylcellulose, polyethylene glycol, waxes and the like.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

Although the active agents of the present method may be administered in divided doses, for example two or three times daily, a single daily dose of each of the HMG-CoA reductase inhibitor and the selective COX-2 inhibitor is preferred, with a single daily dose of both agents in a single pharmaceutical composition being most preferred.

The instant invention also encompasses a process for preparing a pharmaceutical composition comprising combining the HMG-CoA reductase inhibitor and the selective COX-2 inhibitor with a pharmaceutically acceptable carrier, as well as the pharmaceutical composition which is made by combining the HMG-CoA reductase inhibitor and the selective COX-2 inhibitor with a pharmaceutically acceptable carrier.

A therapeutically effective amount of an HMG-CoA reductase inhibitor and a selective COX-2 inhibitor can be used together for the preparation of a medicament useful for preventing Alzheimer's disease, reducing the risk of Alzheimer's disease, delaying the onset of Alzheimer's disease and/or treating Alzheimer's disease or cognitive impairment. For example, the medicament may be comprised of a selective COX-2 inhibitor in combination with about 1 mg to 200 mg of an HMG-CoA reductase inhibitor, or more particularly about 5 mg to 160 mg of the HMG-CoA reductase inhibitor. More specific amounts of HMG-CoA reductase inhibitor which may be used in the medicament preparation include 1 mg, 5 mg, 10 mg, 20 mg, 40 mg, 80 mg, and 160 mg, as well as sub-milligram amounts of HMG-CoA reductase inhibitor's which have sufficient potency at such levels. As a further example, the medicament may be comprised of an HMG-CoA reductase inhibitor in combination with about 0.1 to 200 mg of a selective COX-2 inhibitor.

The instant invention also encompasses the use of an HMG-CoA reductase inhibitor for the preparation of a medicament for the combined use with a selective cyclooxygenase-2 inhibitor for preventing Alzheimer's disease, reducing the risk of Alzheimer's disease, delaying the onset of Alzheimer's disease and/or treating Alzheimer's disease. The medicament or pharmaceutical combination comprised of the HMG-Co reductase inhibitor and the COX-2 inhibitor may also be prepared with one or more additional active agents, such as those described supra.

Examples of dosage formulations suitable for use in practicing the instant invention follow.

### EXAMPLE 1

| Ingredient | Amount per tablet |
|---|---|
| Simvastatin | 5.0 mg |
| BHA | 0.02 mg |
| Ascorbic acid | 2.50 mg |
| Citric acid | 1.25 mg |
| Microcrystalline cellulose | 5.0 mg |
| Pregel starch | 10.0 mg |
| Magnesium stearate | 0.5 mg |
| Lactose | 74.73 mg |

All the ingredients except magnesium stearate are blended together in a suitable mixer. The powder mixture is then granulated with adequate quantities of granulating solvent(s). The wet granulated mass is dried in a suitable dryer. The dried granulation is sized through a suitable screen. The sized granulation is mixed with magnesium stearate before tableting. The tablets may be coated if deemed necessary. Additional ingredients that may be added to the above include suitable color and mixtures of colors.

| Ingredient | Amount per tablet |
|---|---|
| Simvastatin | 5.0 mg |
| BHA | 0.04 mg |
| Citric acid | 2.5 mg |
| Microcrystalline cellulose | 10.0 mg |
| Pregel starch | 20.0 mg |
| Magnesium stearate | 1.0 mg |
| Lactose | 148.46 mg |
| Hydrolized gelatin | 8.0 mg |

The process of manufacture is essentially the same as in Example 1, above.

### EXAMPLE 3

| Ingredient | Amount per tablet |
|---|---|
| Simvastatin | 80.0 mg |
| BHA | 0.16 mg |
| Ascorbic acid | 20.0 mg |
| Citric acid | 10.0 mg |
| Microcrystalline cellulose | 40.0 mg |
| Pregel starch | 80.0 mg |
| Lactose | 550.0 mg |
| Colorant | 5.0 mg |
| Magnesium stearate | 4.8 mg |

The process of manufacture is essentially the same as in Example 1, above.

### EXAMPLE 4

Wet granulated tablet composition

| Amount per tablet | | Ingredient |
|---|---|---|
| 25 | mg | COX-2 Inhibitor |
| 79.7 | mg | Microcrystalline cellulose |
| 79.7 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

Tablet dose strengths of between 5 and 125 mg can be accomodated by varying total tablet weight, and the ratio of the first three ingredients. Generally it is preferable to maintain a 1:1 ratio for microcrystalline cellulose:lactose monohydrate.

### EXAMPLE 4A

Wet granulated tablet composition

| Amount per tablet | | Ingredient |
|---|---|---|
| 12.5 | mg | COX-2 Inhibitor |
| 86 | mg | Microcrystalline cellulose |
| 86 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

### EXAMPLE 4B

Wet granulated tablet composition

| Amount per tablet | | Ingredient |
|---|---|---|
| 10 | mg | COX-2 Inhibitor |
| 87.2 | mg | Microcrystalline cellulose |
| 87.2 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

### EXAMPLE 4C

Wet granulated tablet composition

| Amount per tablet | | Ingredient |
|---|---|---|
| 5 | mg | COX-2 Inhibitor |
| 89.7 | mg | Microcrystalline cellulose |
| 89.7 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

### EXAMPLE 5

Directly compressed tablet composition

| Amount per tablet | | Ingredient |
|---|---|---|
| 25 | mg | COX-2 Inhibitor |
| 106.9 | mg | Microcrystalline cellulose |
| 106.9 | mg | Lactose anhydrate |
| 7.5 | mg | Croscarmellose sodium |
| 3.7 | mg | Magnesium stearate |

Tablet dose strengths of between 5 and 125 mg can be accomodated by varying total tablet weight, and the ratio of the first three ingredients. Generally it is preferable to maintain a 1:1 ratio for microcrystalline cellulose:lactose monohydrate.

### EXAMPLE 5A

Directly compressed tablet composition

| Amount per tablet | Ingredient |
|---|---|
| 12.5 mg | COX-2 Inhibitor |
| 113.2 mg | Microcrystalline cellulose |
| 113.2 mg | Lactose anhydrate |
| 7.5 mg | Croscarmellose sodium |
| 3.7 mg | Magnesium stearate |

### EXAMPLE 5B

Directly compressed tablet composition

| Amount per tablet | Ingredient |
|---|---|
| 10 mg | COX-2 Inhibitor |
| 42.5 mg | Microcrystalline cellulose |
| 42.5 mg | Lactose anhydrate |
| 4 mg | Croscarmellose sodium |
| 1 mg | Magnesium stearate |

### EXAMPLE 5C

Directly compressed tablet composition

| Amount per tablet | Ingredient |
|---|---|
| 5 mg | COX-2 Inhibitor |
| 45 mg | Microcrystalline cellulose |
| 45 mg | Lactose anhydrate |
| 4 mg | Croscarmellose sodium |
| 1 mg | Magnesium stearate |

### EXAMPLE 6

Hard gelatin capsule composition

| Amount per capsule | Ingredient |
|---|---|
| 25 mg | COX-2 Inhibitor |
| 37 mg | Microcrystalline cellulose |
| 37 mg | Lactose anhydrate |
| 1 mg | Magnesium stearate |
| 1 capsule | Hard gelatin capsule |

Capsule dose strengths of between 1 and 50 mg can be accomodated by varying total fill weight, and the ratio of the first three ingredients. Generally it is preferable to maintain a 1:1 ratio for microcrystalline cellulose:lactose monohydrate.

### EXAMPLE 7

Oral solution

| Amount per 5 mL dose | Ingredient |
|---|---|
| 50 mg | COX-2 Inhibitor |
| to 5 mL with Polyethylene oxide 400 | |

Solution dose strengths of between 1 and 50 mg/5 mL can be accomodated by varying the ratio of the two ingredients.

### EXAMPLE 8

Oral suspension

| Amount per 5 mL dose | Ingredient |
|---|---|
| 101 mg | COX-2 Inhibitor |
| 150 mg | Polyvinylpyrrolidone |
| 2.5 mg | Poly oxyethylene sorbitan mono- |
| | laurate |
| 10 mg | Benzoic acid |
| to 5 mL with sorbitol solution (70%) | |

Suspension dose strengths of between 1 and 50 mg/5 ml can be accomodated by varying the ratio of the first two ingredients.

### EXAMPLE 9

Intravenous infusion

| Amount per 200 mL dose | Ingredient |
|---|---|
| 1 mg | COX-2 inhibitor |
| 0.2 mg | Polyethylene oxide 400 |
| 1.8 mg | Sodium chloride |
| to 200 mL | Purified water |

What is claimed is:

1. A method of preventing Alzheimer's disease, reducing the risk of Alzheimer's disease, delaying the onset of Alzheimer's disease and/or treating Alzheimer's disease comprising administering to a patient in need of such treatment a combination of an HMB-CoA reductase inhibitor and a selective inhibitor of COX-2.

2. The method of claim 1 wherein the selectivity of the COX-2 inhibitor is at least 5 fold, as measured by the ratio of the IC₅₀ for the inhibition of COX-1 divided by the IC₅₀ for the inhibition of COX-2.

3. The method of claim 2 wherein the HMG-CoA reductase inhibitor is selected from lovastatin, simvastatin, pravastatin, fluvastatin, atorvastatin, cerivastatin and the pharmaceutically acceptable salt, ester and lactone forms thereof.

4. The method of claim 3 wherein the HMG-CoA reductase inhibitor is selected from lovastatin and simvastatin.

5. The method of claim 3 wherein the HMG-CoA reductase inhibitor is simvastatin.

6. The method of claim 2 wherein the COX-2 inhibitor is selected from:
5-chloro-3-(4-(methylsulfonyl)phenyl)-2-(2-methyl-5-pyridinyl)pyridine;
5(S)-5-ethyl-5-methyl-4-(4-(methylsulfonyl)phenyl)-3-(2-propoxy)-5H-furan-2-one;
3-phenyl-4-(4-(methylsulfonyl)phenyl)-2-(5H)-furanone;
4-[5-methyl-3-phenyl-isoxazol-4-yl]benenesulfonamide;
N-[[4-(5-methyl-3-phenylisoxazol-4-yl)phenyl]sulfonyl]propanamide, and
4-[5-(4-methylphenyl)-3-(trifluoromethyl)-1H-pyrazol-1-yl]benzenesulfonamide.

7. The method of claim 6 wherein the COX-2 inhibitor is selected from:
5-chloro-3-(4-(methylsulfonyl)phenyl)-2-(2-methyl-5-pyridinyl)pyridine;
3-phenyl-4-(4-(methylsulfonyl)phenyl)-2-(5H)-furanone;

8. The method of claim 7 wherein the COX-2 inhibitor is 3-phenyl-4-(4-(methylsulfonyl)phenyl)-2-(5H)-furanone.

9. The method of claim 7 wherein the COX-2 inhibitor is 3-phenyl-4-(4-(methylsulfonyl)phenyl)-2-(5H)-furanone the HMG-CoA reductase inhibitor is simvastatin.

10. A method of claim 2 of preventing Alzheimer's disease, comprising administering to a patient in need of such treatment a combination of an HMB-CoA reductase inhibitor and a selective inhibitor of COX-2.

11. The method of claim 10 wherein the HMG-CoA reductase inhibitor is selected from lovastatin, simvastatin, pravastatin, fluvastatin, atorvastatin, cerivastatin and the pharmaceutically acceptable salt, ester and lactone forms thereof.

12. The method of claim 11 wherein the HMG-CoA reductase inhibitor is selected from lovastatin and simvastatin.

13. The method of claim 12 wherein the HMG-CoA reductase inhibitor is simvastatin.

14. The method of claim 10 wherein the COX-2 inhibitor is selected from:
5-chloro-3-(4-(methylsulfonyl)phenyl)-2-(2-methyl-5-pyridinyl)pyridine;
5(S)-5-ethyl-5-methyl-4-(4-(methylsulfonyl)phenyl)-3-(2-propoxy)-5H-furan-2-one;
3-phenyl-4-(4-(methylsulfonyl)phenyl)-2-(5H)-furanone;
4-[5-methyl-3-phenyl-isoxazol-4-yl]benenesulfonamide;
N-[[4-(5-methyl-3-phenylisoxazol-4-yl)phenyl]sulfonyl]propanamide, and
4-[5-(4-methylphenyl)-3-(trifluoromethyl)-1H-pyrazol-1-yl]benzenesulfonamide.

15. The method of claim 14 wherein the COX-2 inhibitor is selected from:
5-chloro-3-(4-(methylsulfonyl)phenyl)-2-(2-methyl-5-pyridinyl)pyridine;
3-phenyl-4-(4-(methylsulfonyl)phenyl)-2-(5H)-furanone;

16. The method of claim 15 wherein the COX-2 inhibitor is 3-phenyl-4-(4-(methylsulfonyl)phenyl)-2-(5H)-furanone.

17. The method of claim 10 wherein the COX-2 inhibitor is 3-phenyl-4-(4-(methylsulfonyl)phenyl)-2-(5H)-furanone the HMG-CoA reductase inhibitor is simvastatin.

18. A method of claim 2 for reducing the risk of Alzheimer's disease, comprising administering to a patient in need of such treatment a combination of an HMB-CoA reductase inhibitor and a selective inhibitor of COX-2.

19. The method of claim 18 wherein the HMG-CoA reductase inhibitor is selected from lovastatin, simvastatin, pravastatin, fluvastatin, atorvastatin, cerivastatin and the pharmaceutically acceptable salt, ester and lactone forms thereof.

20. The method of claim 19 wherein the HMG-CoA reductase inhibitor is selected from lovastatin and simvastatin.

21. The method of claim 20 wherein the HMG-CoA reductase inhibitor is simvastatin.

22. The method of claim 18 wherein the COX-2 inhibitor is selected from:
5-chloro-3-(4-(methylsulfonyl)phenyl)-2-(2-methyl-5-pyridinyl)pyridine;
5(S)-5-ethyl-5-methyl-4-(4-(methylsulfonyl)phenyl)-3-(2-propoxy)-5H-furan-2-one;
3-phenyl-4-(4-(methylsulfonyl)phenyl)-2-(5H)-furanone;
4-[5-methyl-3-phenyl-isoxazol-4-yl]benenesulfonamide;
N-[[4-(5-methyl-3-phenylisoxazol-4-yl)phenyl]sulfonyl]propanamide, and
4-[5-(4-methylphenyl)-3-(trifluoromethyl)-1H-pyrazol-1-yl]benzenesulfonamide.

23. The method of claim 22 wherein the COX-2 inhibitor is selected from:
5-chloro-3-(4-(methylsulfonyl)phenyl)-2-(2-methyl-5-pyridinyl)pyridine;
3-phenyl-4-(4-(methylsulfonyl)phenyl)-2-(5H)-furanone;

24. The method of claim 23 wherein the COX-2 inhibitor is 3-phenyl-4-(4-(methylsulfonyl)phenyl)-2-(5H)-furanone.

25. The method of claim 18 wherein the COX-2 inhibitor is 3-phenyl-4-(4-(methylsulfonyl)phenyl)-2-(5H)-furanone the HMG-CoA reductase inhibitor is simvastatin.

26. A method of claim 2 for delaying the onset of Alzheimer's disease comprising administering to a patient in need of such treatment a combination of an HMB-CoA reductase inhibitor and a selective inhibitor of COX-2.

27. The method of claim 26 wherein the HMG-CoA reductase inhibitor is selected from lovastatin, simvastatin, pravastatin, fluvastatin, atorvastatin, cerivastatin and the pharmaceutically acceptable salt, ester and lactone forms thereof.

28. The method of claim 27 wherein the HMG-CoA reductase inhibitor is selected from lovastatin and simvastatin.

29. The method of claim 28 wherein the HMG-CoA reductase inhibitor is simvastatin.

30. The method of claim 26 wherein the COX-2 inhibitor is selected from:
5-chloro-3-(4-(methylsulfonyl)phenyl)-2-(2-methyl-5-pyridinyl)pyridine;
5(S)-5-ethyl-5-methyl-4-(4-(methylsulfonyl)phenyl)-3-(2-propoxy)-5H-furan-2-one;
3-phenyl-4-(4-(methylsulfonyl)phenyl)-2-(5H)-furanone;
4-[5-methyl-3-phenyl-isoxazol-4-yl]benenesulfonamide;
N-[[4-(5-methyl-3-phenylisoxazol-4-yl)phenyl]sulfonyl]propanamide, and
4-[5-(4-methylphenyl)-3-(trifluoromethyl)-1H-pyrazol-1-yl]benzenesulfonamide.

31. The method of claim 30 wherein the COX-2 inhibitor is selected from:
5-chloro-3-(4-(methylsulfonyl)phenyl)-2-(2-methyl-5-pyridinyl)pyridine;
3-phenyl-4-(4-(methylsulfonyl)phenyl)-2-(5H)-furanone;

32. The method of claim 31 wherein the COX-2 inhibitor is 3-phenyl-4-(4-(methylsulfonyl)phenyl)-2-(5H)-furanone.

33. The method of claim 26 wherein the COX-2 inhibitor is 3-phenyl-4-(4-(methylsulfonyl)phenyl)-2-(5H)-furanone the HMG-CoA reductase inhibitor is simvastatin.

34. A method of claim 2 for treating Alzheimer's disease, comprising administering to a patient in need of such treatment a combination of an HMB-CoA reductase inhibitor and a selective inhibitor of COX-2.

35. The method of claim 34 wherein the HMG-CoA reductase inhibitor is selected from lovastatin, simvastatin, pravastatin, fluvastatin, atorvastatin, cerivastatin and the pharmaceutically acceptable salt, ester and lactone forms thereof.

36. The method of claim 35 wherein the HMG-CoA reductase inhibitor is selected from lovastatin and simvastatin.

37. The method of claim 36 wherein the HMG-CoA reductase inhibitor is simvastatin.

38. The method of claim 34 wherein the COX-2 inhibitor is selected from:
5-chloro-3-(4-(methylsulfonyl)phenyl)-2-(2-methyl-5-pyridinyl)pyridine;
5(S)-5-ethyl-5-methyl-4-(4-(methylsulfonyl)phenyl)-3-(2-propoxy)-5H-furan-2-one;
3-phenyl-4-(4-(methylsulfonyl)phenyl)-2-(5H)-furanone;
4-[5-methyl-3-phenyl-isoxazol-4-yl]benenesulfonamide;
N-[[4-(5-methyl-3-phenylisoxazol-4-yl)phenyl]sulfonyl]propanamide, and
4-[5-(4-methylphenyl)-3-(trifluoromethyl)-1H-pyrazol-1-yl]benzenesulfonamide.

39. The method of claim 38 wherein the COX-2 inhibitor is selected from:
5-chloro-3-(4-(methylsulfonyl)phenyl)-2-(2-methyl-5-pyridinyl)pyridine;
3-phenyl-4-(4-(methylsulfonyl)phenyl)-2-(5H)-furanone;

40. The method of claim 39 wherein the COX-2 inhibitor is 3-phenyl-4-(4-(methylsulfonyl)phenyl)-2-(5H)-furanone.

41. The method of claim 34 wherein the COX-2 inhibitor is 3-phenyl-4-(4-(methylsulfonyl)phenyl)-2-(5H)-furanone the HMG-CoA reductase inhibitor is simvastatin.

41. Use of a combination of an HMB-CoA reductase inhibitor and a selective inhibitor of COX-2 in the manufacture of a medicament for preventing Alzheimer's disease, reducing the risk of Alzheimer's disease, delaying the onset of Alzheimer's disease and/or treating Alzheimer's disease.
